(19) United States
(12) Patent Application Publication
(10) Pub. No.: US 2004/0259246 A1
(43) Pub. Date: Dec. 23, 2004

(54) Liver cell progenitor and use for treatment of liver diseases

(76) Inventors: Amar Paul Dhillon, London (GB); Mark William Lowdell, London (GB)

Correspondence Address: NIXON & VANDERHYE, PC, 1100 N GLEBE ROAD, 8TH FLOOR, ARLINGTON, VA, 22201-4714, US

(21) Appl. No.: 10/483,190

(22) PCT Filed: Jul. 10, 2002
(86) PCT No.: PCT/GB02/03160

(30) Foreign Application Priority Data
Jul. 10, 2001 (GB) 0116842.6
Mar. 19, 2002 (GB) 0206500.1

(51) Int. Cl. C12N 5/08

(52) U.S. Cl. 435/370; 435/372

(57) ABSTRACT

This invention relates to the generation of liver cells and in particular to the repopulation of the liver with healthy cells for patients with liver disease, especially acute liver failure. The invention thus provides a human liver cell progenitor characterised by the following markers: (a) CD 117 positive, and (b) CD 133 positive, and (c) CD 34 negative.

### FIELD OF THE INVENTION

This invention relates to the generation of human liver cells, and in particular to the repopulation of the liver with healthy cells for patients with liver disease, especially, but not exclusively acute liver failure.

### BACKGROUND TO THE INVENTION

Patients with acute liver failure, who are those most exposed to shortage of donor organs, may particularly benefit from liver cell transplantation. Data obtained from the European Liver Transplant Registry, which includes data from 114 transplant centres in 19 European countries, show that in the period from 1988 to 1998, 2615 patients were transplanted for acute liver failure (10% of the total number of patients transplanted in the same period).

The concept of liver regeneration in human adult liver has been revolutionised in the last decades with the discovery of a hepatic stem cell compartment which is able to regenerate hepatocytes and biliary epithelial cells after hepatic injury. It has previously been shown that, after bone marrow transplantation, hepatocytes of donor origin appear in host liver, indicating that allogeneic liver-cell-precursors of extrahepatic origin can contribute to the liver (Nature 2000:406:257). Phenotypic and functional characterisation of liver cell precursors have therefore become major priorities in the fields of tissue engineering, liver support technology and gene therapy.

The object of the present invention is to establish a system to isolate and expand stem cells which may be used for therapeutic liver re-population in patients with liver disease. It is furthermore our belief that access to autologous or allogeneic hepatic stem cells would allow the development of an “artificial liver”.

U.S. Pat. No. 5,843,633 (also International application WO97/41224) describes the use of a monoclonal antibody AC133 which binds to CD133, a surface antigenic marker for a sub-set of haematopoietic progenitor cells which are found in bone marrow and are CD34 positive. Antibody AC133 is intended for use in the isolation of haematopoietic stem/precursor cells i.e. cells destined to become blood cells.

International patent publication WO 00/43498 describes immunoselection methods to isolate and preserve human hepatic progenitors and identifies a novel hepatic stern cell phenotype which expresses CD34.

### SUMMARY OF THE INVENTION

In studies on diseased adult livers we have found that repopulation of liver cells is taking place from a sub-set of liver cell progenitors which we have now for the first time isolated and characterised. These cells differ from those to which the two above mentioned publications relate, and they are characterised by the following markers: CD34 negative, CD117 positive, and CD133 positive. This specific combination of cell surface molecules expressed on hepatocyte stem or progenitor cells is unique to that cell type and can be used to isolate the cells for ex-vivo culture and expansion to produce non-haematopoietic cells.

The present invention therefore comprises a human liver cell progenitor characterised by the following markers:

(a) CD 117 positive

(b) CD 133 positive

(c) CD 34 negative.

The invention also comprises a population of human liver cell progenitors having the characteristics defined above. Such a population may be derived from any source of stem cells, including the adult liver or bone marrow, by fractionation methods based on the use of the above defined markers. A population obtained in this way may be described as having an “enriched proportion” of the newly identified cells, which term signifies that the cell fraction of interest has a higher proportion of the specified cells than the source material from which the fraction has been derived. This includes a fraction separated from source material to any desired degree of purity with respect to those cells having the markers specified below according to the present invention. Complete isolation of the desired cells from all other cells with which they were originally associated is the preferred degree of enrichment for clinical and related applications.

The invention also embraces populations of cells having the novel defined markers and in combination with a maintenance culture medium or produced by culture of the original fraction obtained from body organs or cells.

The present invention also includes any method of separating from a stem cell source a fraction of non-haematopoietic liver cell progenitors characterised by the following markers:

(a) CD 117 positive

(b) CD 133 positive

(c) CD 34 negative.

This may be achieved with the use of monoclonal antibodies to the specified markers, applied in series or in combination.

The invention also provides a method of maintaining a cell in culture comprising:

(a) providing a cell having the characteristic marker combination of the invention in a culture medium, and

(b) causing said cell to divide or differentiate, or to divide and differentiate.

The invention also provides medical applications of cells having the marker combination of the invention and of differentiated cells derived therefrom.

### BRIEF DESCRIPTION OF THE DRAWINGS

FIG. 1: Acute liver failure—areas of collapse containing proliferating bile ductules and associated small lymphoid-like cells around a portal tract (centre of picture).

FIG. 2: (A) Acute liver failure—scattered cells in areas of bile ductular proliferation show expression of CD 117. (B) Expression of CD34 is seen in endothelial cells only.

FIG. 3: Double epitope immunofluorescence: Staining for CD117 (c-kit) with FITC and mast for cell tryptase with TRITC. Progenitor cells express only CD117.

FIG. 4: Flow cytometric analysis of (A) hepatocytes and (B) non-parenchymal cell fractions from disaggregated liver showing presence of CD34−/CD117+ population (R2) in the NPC fraction.

FIG. 5; CD117+/CD34− ve/haematopoietic marker −ve cells isolated from the non-parenchymal (NPC) fraction of explant livers. These two cells have been isolated from the NPC fraction of an explant adult human liver and labelled with an antibody to CD117 which carries a red fluorescent dye. They have been simultaneously labelled with a cocktail of antibodies to mature and immature haematopoietic cells, including anti-CD34, which each carry a green fluorescent dye. The cell on the left shows expression of CD117 as a red signal in the cytoplasm and on the membrane. It lacks any green signal and is therefore CD34−ve and not of haematopoietic origin. In contrast, the cell on the right shows cytoplasmic and cell surface labelling with both red and green signals and it is thus of haematopoietic origin.

FIG. 6: FACS analysis demonstrates co-expression of CD117 and CD 133, in a CD34 negative cell population.

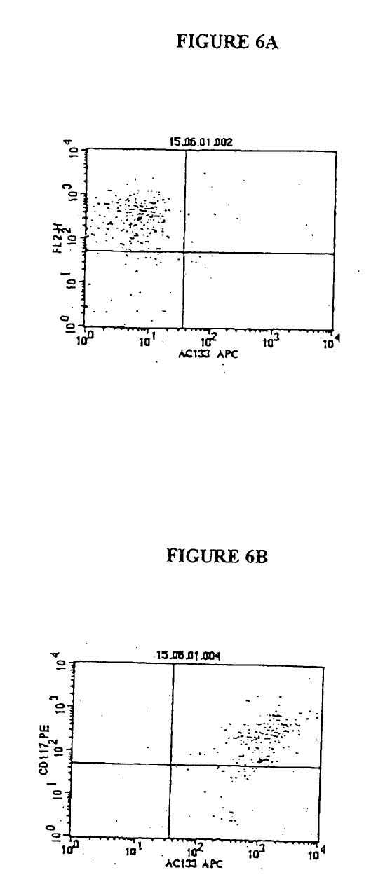

### DETAILED DESCRIPTION OF THE INVENTION

Definitions

In the following description, the meanings of certain technical terms used are given below:

STEM CELLS: Primitive pluripotent cells capable of self-renewal and differentiation into progenitor cells of more than one lineage.

HEPATIC PROGENITOR CELLS: Hepatic progenitor cells refer to stem cells or progeny of stem cells which do not yet express all characteristics of differentiated cell populations of the liver, but have the capacity to express characteristics of one or more differentiated subpopulations of liver cells. For example, but not exclusively, they may have the capacity to differentiate into hepatocyte and/or biliary epithelial cells.

HAEMATOPOIETIC PROGENITOR CELLS: Primitive cells derived from a stem cell precursor which are capable of differentiation into more mature haematopoietic cells in one or more lineages dependent upon their stage of differentiation.

Progenitor Cells of the Invention

Progenitor cells of the invention are hepatic progenitor cells as defined above.

In terms of immunohistochemical properties, they are characterised by the following markers.

(a) CD 117 positive, and

(b) CD 133 positive, and

(c) CD 34 negative.

We have found that CD 133 co-expresses with CD117 in these cells.

Preferably, they are further characterised by one or more of the following additional markers.

(d) CD45 negative, and/or

(e) mast cell tryptase negative

(f) lin negative.

Typically, cells of the invention are of human origin, though it is envisaged that corresponding non-human cells can be isolated from animals such as rodents, e.g. rats and mice; and other animals including cats, dogs, pigs, sheep, horses and cows.

In terms of histology, hepatic progenitor cells of the invention are typically round or oval in shape. Maximum cellular diameter is typically from 5 to 15 μm, preferably from 6 to 12 μm, 6 to 10 μm or 7 to 12 μm. Their cytoplasm is normally scanty, or they contain a moderate amount of amphophilic cytoplasm. They are non-endothelial, non-hepatocyte and non-biliary cells and may have a lymphoblastoid, immunoblastic or plasmacytoid appearance, e.g. as judged by the appearance of the Golgi apparatus. They are found in the non-parenchymal (NPC) fraction of liver cell perfusates (see below for discussion of perfusion methods).

Compositions Comprising Progenitor Cells of the Invention

Progenitor cells of the invention will generally be in isolated form. Typically, they will be comprised in a composition comprising a population of such cells, and a maintenance or culture medium. Any suitable medium may be used (see below).

In such a composition, the proportion of progenitor cells of the invention will typically be enriched. A composition is enriched in progenitor cells of the invention if it comprises a higher proportion of progenitor cells of the invention than in the original sample from which it was obtained, e.g. a higher proportion of progenitor cells of the invention than a explant liver perfusate obtained according to standard techniques such as cannulation followed by perfusion with digestive enzymes such as collagenase or dispase or hyaluronidase, or combinations thereof (e.g. as described below), or than the non-parenchymal cell (NPC) fraction of such a perfusate.

Ideally, in such a composition, all the cells will be progenitor cells having the characteristic marker pattern of the invention. However, even with modern separation techniques such as fluorescence activated cell sorting (FACS), less than total homogeneity may be obtained. Thus, the invention also extends to compositions wherein 10% or more, 25% or more, 50% or more, 60% or more, 70% or more, 80% or more, 90% or more, 95% or more, 99% or more, 99.5% or more, or 99.9% or more of the cells have the marker pattern of the invention.

Isolation of Progenitor Cells of the Invention

Methods for the isolation of the desired stem cell fraction are described in detail hereinafter. Any convenient source of stem cells can be used from which the desired fraction can be separated by use of the markers specified hereinbefore, using standard methods including, but not restricted to, flow cytometric and immunomagnetic methods. Both adult liver and bone marrow are defined sources for the purposes of the present invention.

Recovery of the Stem/Progenitor Cells of the Invention

Any single cell suspension containing more than 0.5% frequency of CD117+/CD34−ve/AC133+ve cells is suitable for the isolation of these cells.

We have used cell suspensions isolated from digests of explant livers recovered from recipients of orthotopic liver transplants. A detailed description of one possible technique is given below.

A segment of liver (150-200 g) is prepared with a single cut surface, from the explant liver. Major venous channels are cannulated with a 14-g cannula. The liver is initially perfused for 5 min with 0.5 mM EGTA-10 mM HEPES-calcium free Hanks balanced salts solution, followed by 5 mins with HEPES-Hanks balanced salts solution and the perfusate collected (pre-digestion perfusate). The segment is then perfused for 10-60 min. with the DMEM with 25 mM EPES containing digestion enzymes (collagenase or dispase or hyaluronidase, or combinations thereof), at 37° C., with a recirculation system such that each cannulated vessel receives a flow rate of ˜30 ml/min. After perfusion cells are collected from the perfusion reservoir (recirculation perfusate), and from the liver fragment after gentle manipulation (disaggregated cell isolate). Further cell digestion is prevented by addition of protease inhibitors, and medium supplemented with anti-oxidants.

Hepatocytes are removed by centrifugation at 50 g in HBSS, and the non-parenchymal cell (NPC) population (supernatant) separated from both hepatocytes and collagenase by differential centrifugation. A 400×“g” centrifugation step of the non-parenchymal cell supernatant removes collagenase. Both pellets are resuspended in HBSS, filtered through a 15 μm filter and re-centrifuged. The non-parenchymal cell pellet is resuspended in HBSS, filtered again through a 15 μm nylon filter and prepared for FACS analysis and sorting by the addition of DNAse (90 Kunitz units). The Hepatocyte pellet is washed twice more and the washes combined and filtered through a 15 μm filter. The final suspensions are centrifuged at 200×“g” to pellet the cells and the pellet resuspended in 0.2-1 ml “sorting buffer” (PBS supplemented with Dnase (90 Kunitz units per ml) and 0.05M EDTA). A sample of pre-sorted cells is taken for FACS analysis to estimate proportion of cells of interest in the population.

Progenitor cells of the invention may then be separated by any suitable technique that discriminates between cells having the marker pattern of the invention and other cell types. Generally, such techniques will be based on the use of antibodies, normally monoclonal antibodies, to the characteristic markers of progenitor cells of the invention. Such techniques use antibodies to bind CD34+ cells, which are discarded, and to bind CD117+ and CD133+ cells, which are retained. The three types of antibody may be used in combination or sequentially. Since CD133 co-expresses with CD117, it may be enough to use antibodies only to CD34 and one of CD 117 and CD133 in some embodiments of the invention.

Two techniques that can be used are indirect magnetic cell sorting, e.g. with microbeads and direct (high purity) cell sorting, e.g. by flow cytometric techniques such as fluorescence activated cell sorting (FACS). Indirect cell sorting may be used as a preliminary step prior to direct cell sorting. Alternatively, direct cell sorting may be used in isolation, without a preliminary indirect sorting step. Whether or not direct cell sorting can be used without preliminary indirect sorting will depend on, for example, the proportion of CD117+/CD34−/CD133+ cells in the source preparation. Detailed descriptions of some possible techniques are given below, though other appropriate techniques may be employed.

For Enrichment by Magnetic Cell Sorting with Indirect Microbeads (MACS Milteny Biotech):

Cells are resuspended in sufficient MACS buffer (PBS, 0.5% BSA, 0.05M EDTA, DNAse 90 units/ml) to obtain a final cell concentration of approximately 10⁸ cells/ml. Anti CD117-PE (for example BD: clone 104D2 although any anti-CD117 monoclonal antibody may be used) is added to cell suspension at 4° C. for 15 mins. Thereafter 5 ml MACS buffer are added, the suspension is centrifuged at 200×“g” for 10 min. The pellet is resuspended in 0.08 ml buffer/10⁸ cells. 0.02 ml anti-PE (phycoerythrin) conjugated MACS paramagentic microbeads (Milteny Biotech) is added and incubated at 4° C. for 30 min. Thereafter the volume is adjusted to 0.5 ml with MACS buffer.

A VS+ or MS+ type MACS column is pre-filled with 1 ml buffer, attached to magnet, and the buffer allowed to pass through. The cell suspension is added to the top of the column; CD 117-negative cells pass through.

The column is rinsed with 3×0.5 ml buffer. 1 ml buffer is then added to the column, the column removed from the magnet, held over a sterile tube and the CD 117-positive cells collected by plunging the buffer through the column with the column plunger provided.

The cells are centrifuged at 200×“g” for 10 min. and resuspended in proliferating medium.

This indirect approach works by addressing one parameter (marker) at a time, i.e. it uses an antibody to one marker at a time. It decreases the numbers of cells in sample from billions (10⁹) to millions (10⁶). Sorting by CD117 marker is illustrated below but the same technique could be applied to CD133 as the cells of the invention are positive for both markers.

For Direct Cell Sorting, Which may be Subsequent High-Purity Cell Sorting Following an Indirect Sort as Described Above or may be Used Without a Preliminary Indirect Sort in Appropriate Cases:

The suspension is incubated with 0.02 ml of each of the following monoclonal antibodies: CD34FITC (e.g: BD clone (anti-HPCA-2) 8G12, although any anti-CD34 monoclonal antibody may be used), CD117PE (e.g: BD clone 104D2 although any anti-CD117 monoclonal antibody may be used) and CD133APC (previously known as AC133) (e.g: Milteny Biotech clone AC133 although any anti-CD133 monoclonal antibody may be used). The suspensions are incubated for 20 min at 21° C. and then washed once by addition of 5 ml “sorting buffer” followed by centrifugation at 200×“g”. Cells are resuspended in 2 ml “sorting buffer” and passed to a conventional stream-in-air cell sorter equipped with a 70 μm sort nozzle and dual laser optics (488 nm and 635 nm sources) for isolation (e.g. FACS Vantage SE, BD Biosciences, Cowley Oxford UK).

Using conventional flow cytometric calibration particles (e.g. Calibrite, BD Biosciences, Cowley Oxford UK) and the sorter electronic threshold set on the forward angle light scatter signal, align the sample fluidics to the laser interception point and focus the emitted FITC, PE and APC fluorescent signals onto their respective photomultiplier tubes (PMTs). Set-up the fluorescence detector voltages to provide optimal fluorescent signals and use the inherent compensation circuitry to account for fluorescent spectral overlap. Using a sample pressure of 12-15 PSI and a sample differential sufficient to achieve a threshold rate of 8-12 K/sec., establish the optimal settings for one-way sorting. Set the drop-drive frequency (DDF) to >25K/sec and adjust the drive amplitude to between 50-80%. Use the phase control to obtain a single stream in “normal Recovery” sort mode with a 2-drop sort window. Calculate the drop-delay and note the position of the break-off drop.

The cells of interest fall between 6 and 10 μm and the forward scatter photodiode voltage and linear amplifiers must be adjusted such that the cells of interest fall between channels 200-600 on a 1024 channel linear scale.

Set-up a 2-D dot plot of FSC versus PE and define a sort region (region 1) on the basis of FSC above linear channel 400 FITC below linear channel 400. In a second 2-D dot plot arrange PE versus APC and define a second sort region (region 2) on the basis of PE fluorescence above linear channel 400 and APC fluorescence above linear channel 400. Define a sort gate (gate 1) of “region 1 AND region 2” and set the right sort gate to “gate 1”. Run the cells through the cell sorter whilst monitoring the stream break-off drop for evidence of instability. Sorted cells should be maintained on ice until placed into culture.

The purity and yield of cells isolated in the manner described above will be determined by a number of factors, not least of which will be the frequency of the target cell population in the initial material and the expertise of the operator of the cell sorter. However, careful selection from a starting population containing 0.05-0.1% CD34−/CD117+/CD133+ cells should render a population which is 80-95% pure with a yield of 30-50%.

Such direct cell sorting techniques can be carried out using FACS apparatus and will generally address all three parameters (markers) at once.

Maintenance and Subculture of Cells of the Invention in Culture

Cells having the characteristic marker pattern of the invention may be maintained in culture in any suitable manner. They may be cultured in such a way as to allow them divide without differentiating (i.e. to self-renew) or in a way that allows them to differentiate. In the latter case, they may or may not divide as well.

The invention extends to cells derived from those having the characteristic marker pattern of the invention by any means, whether or not they share the marker pattern. Thus, further progenitor cells derived from cells with the marker pattern of the invention are provided. These will typically be obtained by division. Also provided are differentiated cells.

Maintenance

For example, semi-purified and FACS sorted cells may be maintained in the following ways:

A) On human placental matrix, fibronectin, collagen, laminin or plastic: cells are plated in a variety of culture media. (i) “Proliferating” medium (alpha MEM containing ribo- and deoxy-ribonucleosides, supplemented with foetal bovine serum, penicillin—streptomycin, fungizone, 25 mM glucose, 5-40 ng/ml Hepatocyte growth factor, 1-20 ng/ml epidermal growth factor, 1-10 ng/ml tumour necrosis factor alpha, ferrous sulphate 0.5 mg/L, Zinc sulphate 0.75 mg/L, 1-10 mM nicotinamide, sodium selenite and free fatty acids, albumin). (ii) serum free medium containing all of the above supplements except foetal bovine serum. Medium is replenished three times per week.

B) In semi-solid methyl cellulose gels cells are resuspended at 10⁶-10⁸/ml in proliferating medium (as defined above). Methyl cellulose is diluted to 0.9% in proliferating medium. Suspended cells (100 μl) are added to 2.9 ml methyl cellulose, mixed, and with a blunt ended needle aliquotted at 1 ml per 35 mm diameter petri dish. After thorough mixing dishes are incubated at 37° C. in a humidified 5% CO₂ incubator for 14 days. Colony forming assays are performed on day 14.

C) On feeder layers of stromal cells: A variety of stromal cells of human and murine origin will be used as feeder cells for culture of human hepatic progenitors. Stromal cells will be irradiated or treated with mitomycin C to prevent overgrowth, prior to seeding with putative progenitor cells.

We have also found that progenitor cells of the invention can be cryopreserved, i.e. frozen. For example, they may be preserved in liquid Nitrogen. Appropriate cryopreservant additives may be used. Thus, the cells may be maintained in a frozen state until required.

Subculture

Subculture may be carried out in any suitable manner. For example, cells may be cultured in artificial media at 33-39° C., under conditions designed to encourage proliferation of hepatic progenitor cells. In particular, sub-culture conditions are designed to encourage proliferation of hepatic progenitor cells which have the capacity to differentiate under appropriate conditions into hepatocytes. In fulfilling the aim of repopulating the liver, the process of differentiation into mature hepatocytes will be completed after administration via the vascular system to patients with liver disease.

In fulfilling the aim of developing a bioartificial liver, culture conditions, after proliferation and expansion of cell populations has been achieved, will predominantly, but not exclusively, be those designed to encourage production of hepatocyte characteristics, in vitro, especially in configurations appropriate to subsequent use in extracorporeal perfusion systems and drug testing.

Such conditions may include culture in alginate or other semi-solid media, culture in or on beads, culture in or on semi-permeable or impermeable chemical polymers, culture in hollow fibre cartridges, culture in multilamellar configurations. Expression of differentiated hepatocyte function will be encouraged by addition of organic fibrillar proteins, such as fibronectin and laminin, and by addition of growth factors and cytokines, including Hepatocyte growth factor, Keratinocyte growth factor, epidermal growth factor, transforming growth factor alpha and beta, nerve growth factor and Insulin like growth factor. Another hepatocyte characteristic that may be observed during this process is expression of albumin. Chemical agents promoting both proliferation and differentiation may also be used.

Conditions designed to encourage differentiation into biliary epithelial cells or other types of liver cell may also be used.

Cells may be cultured for any suitable time, e.g. periods of days, weeks, months or years, in order to acquire suitable characteristics for use as described below. For example, cells may be culture for up to one day, up to one week, up to two weeks, up to one month, up to two months, up to six months or up to one or two years.

During the process of differentiation, it is expected that the characteristic marker pattern of the cells of the invention will be lost. It will be appreciated that the CD34−/CD117+/CD133+ combination is characteristic of hepatic progenitor cells of the invention rather than of differentiated cells derived therefrom, which may themselves be indistinguishable from the patient's pre-existing liver cells. Cells that are derived from progenitor cells of the invention but have lost the marker combination during differentiation, e.g. in the direction of hepatocytes, are also aspects of the invention. Further progenitor cells obtained, e.g. by division from cells with the characteristic marker pattern are also an aspect of the invention. Thus, in addition to providing hepatic progenitor cells with the characteristic marker pattern, the invention also provides cells obtained, or obtainable from the hepatic progenitor cells of the invention.

Uses of Cells According to the Invention

Cells of the invention may be used in the treatment of liver disease. Typically, it will be differentiated cells derived from the hepatic progenitor cells of the invention (see “Subculture” above) that are used in treatments rather than the progenitor cells themselves. However, progenitor cells may be used in appropriate cases.

It is envisaged that any condition wherein the patient suffers from reduced liver function can be treated. This includes acute and chronic liver failure and hepatic necrosis and inborn conditions in which metabolic errors affect the liver. Such conditions may be autoimmune conditions.

To put the following in perspective, it should be noted that existing treatments have a number of drawbacks. Liver transplants are available but, in cases of hepatic failure, the survival rate is only around 50% after one year, an entire liver lobe is required for the transplant and the patient must be kept on anti-rejection drugs for a long period. Elective transplants have a lower mortality rate but the waiting list for such transplants is too long for many patients to benefit. Treatments that render traditional transplants unnecessary would therefore be of great value. In addition, the fact that the liver (unlike organs such as the kidney) has great potential for self-regeneration is important. Treatments that reduce the burden on the remaining liver cells of a patient for long enough for self-regeneration to replace lost capacity would also be of great benefit.

According to the invention, several particular types of treatment are envisaged, although the invention is not confined to these types of treatment.

Liver Repopulation

Cells of the invention may be used to repopulate the liver. Such cells may be autologous, i.e. derived from the patient's own liver (or that of an identical sibling), in which case no issues of tissue rejection arise. Alternatively, they may be allogeneic, i.e. derived from the liver of another individual. In the latter case, the cells will typically be derived from the liver of a donor individual with the same blood group as the recipient patient and anti-rejection drugs may be needed, as for known types of liver transplant.

Such an approach could be used instead of or alongside a conventional liver transplant. The cells may be the patient's own cells, removed, cultured and replaced, or they may be cells from another individual, as discussed above. For example, they might be the patient's own cells in a chronic condition where there is time to remove and culture them without risking the patient's life (and such an approach may also be used in conjunction with liver dialysis: see below). In acute cases, pre-cultured, non-patient cells may be preferred.

In this connection, cells may be implanted in or on a biocompatible substrate such as a three-dimensional matrix or a membrane. Any suitable material may be used as a substrate. Alginate is one possible example.

Liver Dialysis

A so-called artificial liver can also be provided. In such an embodiment, differentiated liver cells of the invention will be kept outside the body and the patient's blood or plasma, preferably plasma, will be cycled over or through them in a manner similar to that used for kidney dialysis, then returned to circulation in the patient's vascular system. Whilst the patient is undergoing dialysis, the burden on his or her remaining liver tissue is reduced and it has the opportunity to regrow. This is an attractive option because it may enable patients to be completely cured by their own natural regenerative processes without the need for any implantation of cells. It would also be inexpensive and medically straightforward compared to conventional transplantation procedures.

Dialysis procedures can also be used together with repopulation treatments of the invention: the patient can be kept alive using dialysis whilst cells are cultured for repopulation procedures

Both repopulation and dialysis treatments of the invention have the advantage that replacement liver tissue can be grown in culture rather than taken from donors. Unless a treatment of the invention is used alongside a conventional transplant, donor tissue on the scale currently required for liver transplantation will be unnecessary and only a biopsy will be required to generate the required amount of cells. Dialysis procedures of the invention and the use of the patient's own cells in repopulation procedures have the added advantage that anti-rejection treatments are not required.

Gene Therapy

Cells of the invention may also be genetically modified by the introduction of a heterologous nucleic acid coding sequence. They can thus be used in methods of gene therapy. For example, a progenitor cell of the invention may be modified, then cultured as described herein prior to re-implantation of cells (either progenitor cells or differentiated cells derived therefrom). Such ex vivo transformation procedures have safety advantages because the cells can be examined ex vivo to confirm that, for example, the transgene bas not been incorporated close to an oncogene, thus rendering the transformed cell oncogenic.

Methods of transformation known in the art may be used. In principle, any gene can be introduced in this manner. Generally, the gene will be one that complements a deficiency in the patient's own liver tissue.

The following Examples illustrate the invention.

### EXAMPLES

We investigated patients with massive hepatic necrosis (of unknown aetiology) using an immunohistochemical panel including CD34, CD117, and CD133 on the explant liver and by flow cytometry and immunofluorescence on explant liver perfusates. Explant livers obtained from patients with massive hepatic necrosis represent a human model to study liver regeneration. The regeneration process has begun in such tissue and the tissue is expected to be rich in progenitor cells when the patient is transplanted after the onset of liver damage. The study of explant livers should allow liver progenitor cells to be characterised phenotypically and functionally. The aim of our study was to investigate the phenotype of progenitor cells in liver of patients with massive hepatic necrosis. These cells may derive from both intra and/or extrahepatic sources, and may have the potential to differentiate into hepatocytes, biliary epithelial cells, or both.

### Example 1

We studied initially the removed liver (explant) of a patient with massive hepatic necrosis (of unknown cause) who was transplanted 3 months after the onset of symptoms. We performed standard histological analysis and single epitope immunohistochemistry for various markers including CD34, CD117 (c-kit) and CD133. A negative result for CD34 was particularly unexpected. Double epitope immunofluorescence for CD133 and CD45 (Becton Dickinson CD45 PerCP, clone 2D1) was also performed. The second part of the study was carried out on the explant liver of a patient with hepatic necrosis due to autoimmune liver disease to confirm if cells with a similar morphology and phenotype were identifiable in the non-parenchymal cell (NPC) fraction of the liver perfusate and to characterise them further using double immunofluorescence and flow cytometry. A segment of liver from each case was cannulated and perfused with digestion enzymes. Isolated cells were collected, fractionated by differential centrifugation and analysed with double and triple epitope immunofluorescence and flow cytometry for expression of CD117, CD34 and haematopoietic lineage markers. Double epitope immunofluorescence for CD117 and mast cell tryptase was also performed.

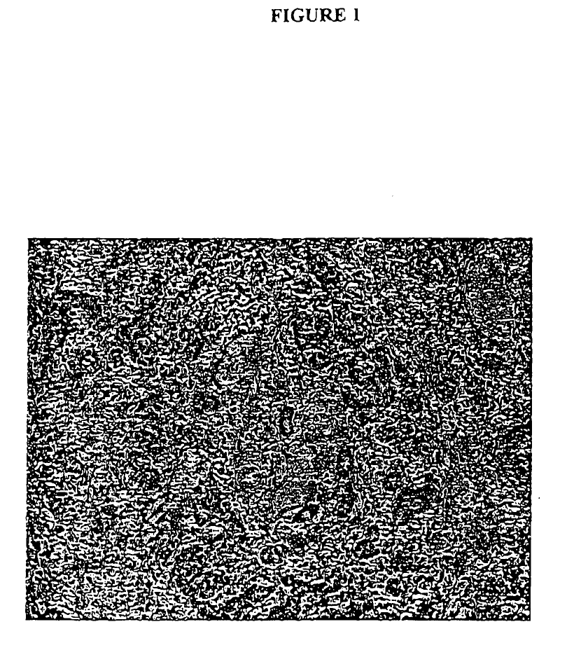

The first case studied showed massive hepatic necrosis histologically. Non-hepatocyte/non-biliary, small, round or oval shaped cells slightly larger than lymphoid cells and with scanty cytoplasm, were associated with regenerating biliary structures (FIG. 1).

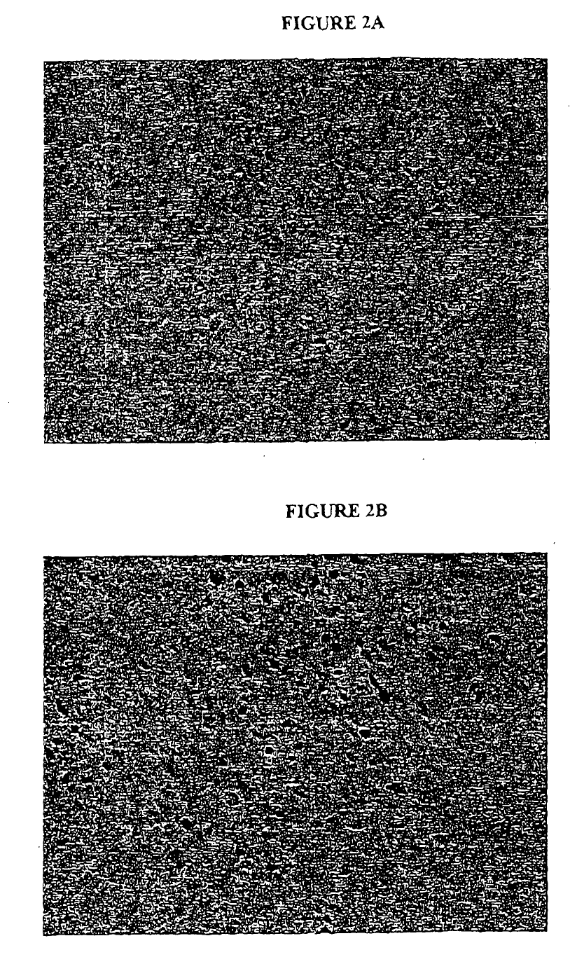

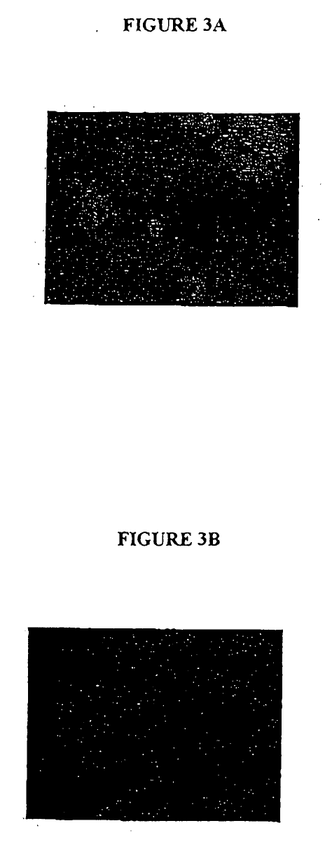

These cells stained with CD117 (in both frozen and paraffin material) but lacked CD34 expression, which was restricted to the vascular endothelial cells (FIG. 2a&b). No non-endothelial CD34 positive cells of this putative progenitor type were identified in the same areas, on either paraffin or frozen material. Double epitope immunofluorescence on liver tissue showed many mast cells expressing both CD117 and tryptase, as well as scanty putative progenitor cells expressing CD117 only (FIG. 3a&b). Double epitope immunofluorescence showed, in the same areas, cells expressing both CD133 and CD45. Some of these cells were of relatively small size and round or oval shape, others were larger with more abundant cytoplasm. Many newly formed biliary structures also showed expression of CD133. Putative hepatic stem/progenitor cell in a portal tract expresses CD117 (green) but lacks mast cell-specific tryptase (red).

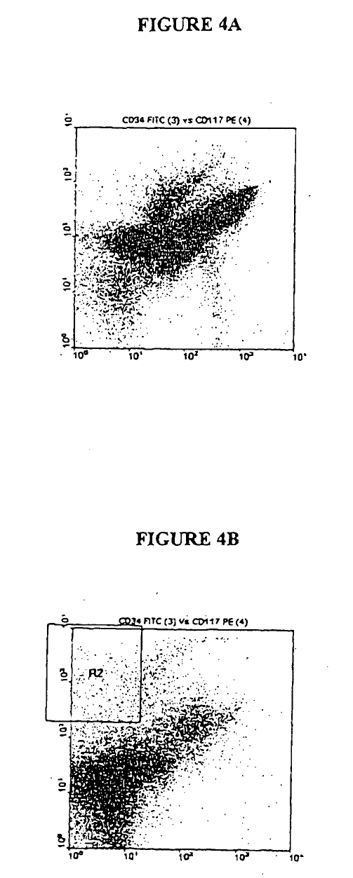

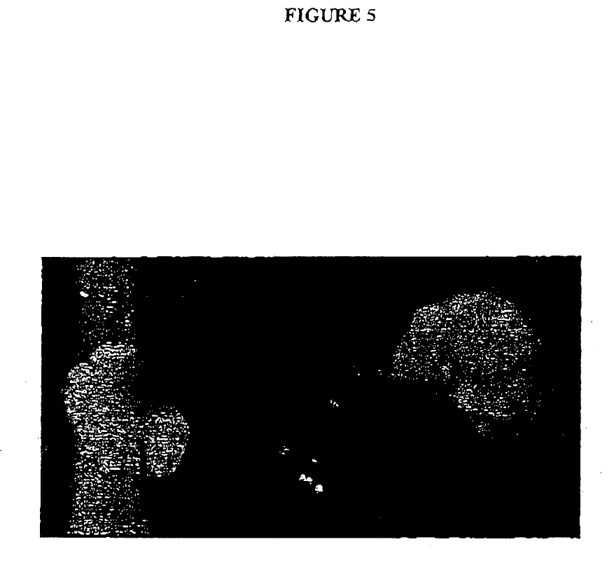

In the second part of the study, double epitope immunofluorescence on the NPC fraction showed CD117+, CD34−ve, Lin−ve (i.e CD3, CD4, CD8, CD14, CD16, CD19, CD33, CD34, CD56) individual cells as well as a separate component of individual cells expressing haematopoietic markers. Flow cytometric analysis identified a CD117+/CD45+/CD34−ve population within the NPC fraction, representing about 3% of the CD45+ cells (FIG. 4) and isolated cells sorted on the basis of CD117 expression were confirmed by double immunofluorescence to lack haematopoietic lineage markers, confirming the primitive nature of the cell population (FIG. 5).

The cell on the left hand side (a putative hepatic stem cell) expresses CD117 (red) in the absence of haematopoietic lineage markers (green). In contrast, the cell on the right expresses both CD117 and lineage markers and is thus a committed haematopoietic progenitor cell.

### Example 2

Further experiments along similar lines were carried out on explants from a number of additional patients.

Materials and Methods

We performed histological analysis and immunohistochemistry for CD34, CD117 (c-kit), CD133, CD45, mast cell tryptase, cytokeratins, and HepPar1. The non-parenchymal cell (NPC) fraction of explant liver perfusates was used to further characterise putative progenitor cells using immunofluorescence and flow cytometry.

We studied six explant livers of patients with massive hepatic necrosis (MHN). The cause of the liver disease was unknown, and investigations for viral hepatitis and autoantibodies were negative. The patients had no known exposure to drugs or hepatotoxins. Fresh tissue was obtained within 1 hour of removal, snap frozen and stored at −80° C. The explant liver was fixed in 10% buffered formalin for 24 hours and samples for paraffin embedding were obtained, subsequently. We performed histological analysis, immunohistochemistry, immunofluorescence, and flow cytometry as described below.

Histological Analysis

This was carried out using a standard haematoxylin and eosin staining.

Immunohistochemistry

Primary antibodies were used as follows: CD34 and CD117 (Dako) were used at 1:50 dilution after microwave pre-treatment in 0.01 citrate buffer, pH6. Mast cell specific tryptase (Novocastra) was used at 1:100 dilution without pre-treatment. Species-specific secondary antibodies were used as appropriate. A standard alkaline phosphatase anti-alkaline phosphatase (APAAP)/Fast Red (Vector Laboratories) method was used. Washing steps were carried out using TBS (pH 7.6) buffer. Sections without secondary antibody were used as negative control. Sections of appendix were used as positive control for CD117 and Mast cell specific tryptase. Sections of placenta were used as positive control for CD34.

Multiple Epitope Immunofluoresence

This was performed to investigate co-expression of CD117, CD34, CD133 and CD45. Paired 10 micron frozen sections of liver were cut and mounted onto slides prior to acetone fixation and then air-dried. Sections on slides were re-hydrated with 1% BSA in PBS, washed and then incubated for 30 min at room temperature with anti-CD133, or mast-cell tryptase Phycoerythrin (PE) (Milteny Biotech, Cologne, Germany) and CD117 or anti-CD45 fluorescein isothiocyanate (FITC, BD, Oxford, UK). Both antibodies were used at a 1:20 dilution. Two minutes before completion of the incubation, 2 drops of H33259 (Sigma, Poole, Dorset) were added to each section as a nuclear counterstain. Sections were washed twice in excess PBS and mounted in anti-fade glycerol-buffered mountant before analysis under epi-illumination fluorescence microscopy.

Flow Cytometry: Cell Retrieval

A segment of liver (150-200 g) was prepared with a single cut surface, from the explant liver. Major venous channels were cannulated with a 14-g cannula. The liver was initially perfused for 5 min with calcium free EGTA-Krebs Henseleit buffer and the perfusate collected (pre-digestion perfusate). The segment was then perfused for 30-60 min. with Krebs-Henseleit solution containing digestion enzymes (collagenase, dispase, hyaluronidase), at 37° C., with a recirculation system such that each cannulated vessel received a flow rate of −30 ml/min. After perfusion cells were collected from the perfusion reservoir (recirculation perfusate), and from the liver fragment after gentle manipulation (disaggregated cell isolate).

Aliquots were kept at each stage of preparation for proportionate analysis. Cells from the disaggregated cell isolate were fractionated by differential centrifugation for sorting. After a 50 g centrifugation to remove the bulk of hepatocytes the supernatant was centrifuged at 400 g for 10 min to pellet the non-parenchymal cell fraction and to remove any remaining collagenase. Washes were performed in HBSS and the final pellet resuspended in HBSS containing 90 units.ml deoxyribonuclease. A ficoll density centrifugation step removed way contaminating red blood cells, prior to analysis by FACS, and subsequent cell sorting.

Dual and Triple Epitope Immunofluorescence of Single Cell Suspensions

Perfusion cells were isolated, fractionated by differential centrifugation and either cytospun onto glass slides (Shandon, UK) or maintained in single cell suspension. Cytospin preparations were labelled for dual epitope immunofluorescence using the same procedure as described above for the dual-parameter analysis of tissue sections. The antibody combinations used on the cytospin preparations were CD34 FITC/CD117 PE and a cocktail of CD3, CD4, CD8, CD14, CD16, CD19, CD33, CD34, CD56 FITC/CD117 PE. Cell suspensions were labelled simultaneously with three fluorochrome-conjugated antibodies and analysed by flow cytometry. Briefly, 10⁵ mononuclear cells were incubated in suspension with 5-10 μl CD34 FITC/CD 117 PEI/CD45 PerCp, washed and analysed by flow cytometry (FACSCalibur, BD, Oxford, UK).

Five bone marrow samples from five normal donors were used as normal controls.

Results

We found non-hepatocyte/non-biliary cells with a lymphoblastoid/immunoblastoid morphology in our cases of MHN. The cellular morphology and distribution altered to some extent with the duration of the hepatic disease. For the purpose of this study we regarded cells as hepatic progenitor cells if they were positive for immunophenotypic marker combinations previously associated with stern cells and/or if they were present in periportal areas or in/adjacent to areas containing proliferating ductules or islands of regenerating hepatocytes cells and did not have the appearance of mature biliary cells or hepatocytes.

We identified cells that were round or oval in shape and 7-12 μm in maximum diameter. They possessed a moderate amount of amphophilic cytoplasm, and some had a Golgi apparatus giving an immunoblastic or plasmacytoid appearance. Rare mitoses were seen in this cell type. Some cells were found scattered within necrotic areas, or within the periportal/portal infiltrate associated with regenerating biliary structures. These cells stained with CD117 (in both frozen and paraffin material). Other cells with different appearance also showed CD117 immunostaining, so that of the CD117+ population, cells of the invention were the minority. The majority of CD117+ cells expressed mast cell specific tryptase. CD117+ progenitor cells co-expressed CD133, and were CD45 negative. No non-endothelial CD34 positive cells of this putative progenitor type were identified on either paraffin or frozen material. Many ductules also showed expression of CD133. CD117 expression was seen in metaplastic (ductular) hepatocytes.

We were thus able to recover up to 10⁶-10⁷ CD117+/CD133+/CD34−ve/Lin−ve cells from the liver explant perfusates. Double epitope immunofluorescence on the NPC fraction showed CD117+/CD34− individual cells as well as a separate component of individual cells expressing haematopoietic markers. Flow cytometric analysis identified a cell population which was CD117+/CD45+/CD34−ve within the NPC fraction and which represented about 3% of the CD45+ cells. Analysis of hepatocytes extracted from the same explant liver showed absence of this cell lineage. Cells with this phenotype were not present in bone marrow samples from five normal donors.

Our flow cytometry results on cells fractionated by differential centrifugation from the liver perfusate support our immunohistochemical findings. The combination of CD117 and CD133 expression and negativity for CD34 has not been described previously and may be used to isolate putative hepatic stem/progenitor cells for further amplification, manipulation and therapeutic use.

Conclusion

We have studied the explant livers obtained from six patients with massive hepatic necrosis. We have identified lymphoblast-like non-hepatocyte/non-biliary/non-mast cell CD117+/CD133+ putative hepatic progenitor cells. CD117+/CD133+ ductules were also seen, i.e. expression of the novel marker CD133 was also identified in the putative progenitor cells and ductules. The presence of a previously unknown hepatic CD117+/CD133+/CD34−ve/lin−ve cell population was confirmed with double immunofluorescence and flow cytometry in the non parenchymal cell fraction from liver perfusates of these cases. No non-endothelial CD34+ cells were identified indicating that intrahepatic adult human progenitor/stern cells have lost (or do not express for some other reason) CD34 expression. The isolation, and enrichment of adult human hepatic progenitor/stem cells for further studies and therapy by CD117/CD133 identification may therefore be preferable to CD34 selection strategies.

1. A non-haematopoietic liver cell progenitor characterised by the following markers:
(a) CD 117 positive, and
(b) CD 133 positive, and
(c) CD 34 negative.

2. A cell of claim 1 further characterised by one or more of the following additional markers:
(a) CD45 negative, and/or
(b) mast cell tryptase negative
(c) lin negative.

3. A cell of claim 1 which has a maximum cellular diameter of from 6 to 12 μm

4. A cell of claim 1 which is a hepatic cell.

5. A cell of 1 which is a bone marrow cell.

6. A cell of claim 1 which is genetically modified by the introduction of a heterologous nucleic acid coding sequence.

7. A cell of claim 1 which is a human cell.

8-11 (canceled)

12. A method of separating from a stem cell source a fraction of non-haematopoietic liver cell progenitors characterised by the following markers:
(a) CD 117 positive
(b) CD 133 positive
(c) CD 34 negative.

13. A method of claim 12 wherein the progenitor cells are human cells.

14. A method of claim 12 wherein the stem cell source is a sample from an adult liver.

15. A method of claim 12 wherein the stem cell source is a bone marrow sample.

16. A method of claims 12, in which separation is achieved using monoclonal antibodies to the specified markers in series or in combination.

17. A method of claim 16 wherein said separation is achieved by a method comprising Fluorescence Activated Cell Sorting (FACS).

18. A method of claim 16 further comprising separating cells on the basis of one or more of the following additional markers:
(a) CD45 negative, and/or
(b) mast cell tryptase negative
(c) lin negative.

19-40 (canceled)

41. A cell derived from a progenitor cell according to claim 1 by division, differentiation or any other process.

42. A cell of claim 41 which is a further progenitor cell, a hepatocyte, a biliary epithelial cell or another type of liver cell.
